(19) United States
(12) Patent Application Publication
(10) Pub. No.: US 2026/0335498 A1
(43) Pub. Date: Oct. 29, 2026

(54) EMBOLIC AGENT

(71) Applicants: STRYKER CORPORATION, Portage, MI (US); STRYKER EUROPEAN OPERATIONS LIMITED, Carrigtwohill (IE)

(72) Inventors: Brent Gerberding, San Jose, CA (US); Hancun Chen, San Ramon, CA (US)

(73) Assignees: STRYKER CORPORATION, Portage, MI (US); STRYKER EUROPEAN OPERATIONS LIMITED, Carrigtwohill (IE)

(21) Appl. No.: 19/699,947

(22) Filed: Jun. 5, 2026

Related U.S. Application Data
(63) Continuation of application No. PCT/US2024/059038, filed on Dec. 6, 2024.
(60) Provisional application No. 63/608,569, filed on Dec. 11, 2023.

(51) Int. Cl. A61L 24/00

(52) U.S. Cl. CPC A61L 24/0089; A61L 24/0036; A61L 2400/04; A61L 2400/06

(57) ABSTRACT

A vaso-occlusive agent for occluding an aneurysm comprises an embolic fluid configured to occlude the aneurysm, and a plurality of AuPt particles suspended within the embolic fluid. At least one of the AuPt particles has a size that is anywhere from 0.1 micron to 100 microns. Additionally, the least one of the AuPt particles comprises a plurality of pores, where the at least one of the pores has a pore size that is anywhere from 1 nanometer to 10 microns. The AuPt particles in the embolic fluid are 10-50% by weight. The AuPt particles in the embolic fluid comprise a volume magnetic susceptibility from −40×10⁻⁶ to 40×10⁻⁶.

### RELATED APPLICATION DATA

This application is a continuation of International Patent Application No. PCT/US2024/059038 filed on Dec. 6, 2024, pending, which claims priority to, and the benefit of U.S. Provisional Patent Application No. 63/608,569 filed on Dec. 11, 2023, lapsed. The entire disclosures of the above applications are expressly incorporated by reference herein.

### FIELD

The present disclosure relates generally to vaso-occlusive agent and intravascular medical procedures, and more particularly, to liquid embolic solutions with enhanced MRI follow-up.

### BACKGROUND

Vaso-occlusive agents are used for a wide variety of reasons, including treatment of intravascular aneurysms. An intravascular aneurysm is a localized, blood-filled, dilation of a blood vessel that typically assumes a sac or balloon-like configuration that extends from a blood vessel and is caused by disease, blood flow/pressure exerted in the vessel, and/or weakening of the vessel wall. Intravascular aneurysms may pose a risk to a patient's health due to rupture, clotting, or dissection, which may cause hemorrhage, stroke (e.g., an intracranial aneurysm) and other damaging consequences to the patient. Approximately 30,000 intracranial aneurysms rupture each year in North America.

There are a variety of approaches to treat a ruptured or non-ruptured aneurysm including (e.g., an endovascular approach that involves delivering vaso-occlusive devices, implants or embolic agents through an endovascular catheter into the aneurysm). A typical endovascular approach for delivering vaso-occlusive agents into an aneurysm includes positioning a small profile delivery catheter or micro-catheter at an aneurysm site using a guidewire. Typically, the distal end of the delivery catheter is provided, either by the attending physician or by the manufacturer, with a selected pre-shaped bend or other shape, depending on the particular anatomy of the patient, so that it will stay in a desired position for releasing the vaso-occlusive agent into the aneurysmal sac. It is desirable that the lumen and the outer diameter of the delivery catheter be as small as possible to allow the aneurysm to be accessed through a very small vasculature. In some embodiments, blood flow to the aneurysm site is blocked (e.g., balloon catheters) during the delivery of vaso-occlusive agents. The vaso-occlusive agent is then released from the distal end of the delivery assembly, and the delivery assembly is withdrawn back through the delivery catheter. Depending on the particular needs of the patient, additional vaso-occlusive devices, implant or agents may be delivered through the delivery catheter and released into the same aneurysmal sac until the aneurysmal sac is completely filled and/or occluded.

Fluroscopy is typically used to visualize vaso-occlusive agents during delivery into an aneurysmal sac, while magnetic resonance imaging (MRI) is typically used to visualize the treatment site post-procedure (e.g., a few weeks after initial treatment of the aneurysm) to ensure that the aneurysmal sac is properly occluded. As such, it is important that vaso-occlusive agents are composed in a manner that enables contrast or radiopacity during treatment of the aneurysm. Also minimizing the magnetic susceptibility of vaso-occlusive agents, such that any visualization obscuring artifacts created during the post-procedure MRI is avoided (i.e., being MRI-compatible).

Commonly used liquid embolic solutions include a mixed of polymers, adhesives, and metallic particles. The polymers and adhesives usually include fast polymerization properties. The most commonly used embolic agent is the fast polymerizing liquid adhesive n-butyl cyanoacrylate (n-BCA). Further, metallic particles added to the liquid embolic solutions include radio-opaque properties for post-procedure visualization. Typically, current embolic solutions contain tantalum powder suspended within the liquid embolic for fluorescent imaging visibility or radiopacity. However, current liquid embolic solutions have exceedingly high sedimentation rate because of the use of larger tantalum particles to achieve radiopacity requirements. In addition, the tantalum powder in liquid embolic produces visualization obscuring artifacts during the post-procedure MRI, which will be described in further detailed below in FIG. 3.

It is important that vaso-occlusive agents and/or liquid embolic solutions are composed in a manner that minimizes any visualization obscuring artifacts created during the post-procedure MRI.

### SUMMARY

A vaso-occlusive agent for occluding an aneurysm includes: an embolic fluid configured to occlude the aneurysm, and a plurality of gold-platinum alloy (AuPt) particles suspended within the embolic fluid.

In some embodiments, the embolic fluid includes a gel, a paste, or a liquid solution. Further, the embolic fluid is composed of biocompatible materials. The biocompatible materials include ethylene vinyl-alcohol copolymer and dimethyl-sulfoxide.

In some embodiments, at least one of the AuPt particles has a size that is anywhere from 0.1 micron to 100 microns. Optionally, the at least one of the AuPt particles includes a plurality of pores, and the at least one of the pores has a pore size that is anywhere from 1 nanometer to 10 microns. Optionally, the at least one of the AuPt particles has a porosity that is anywhere from 1% to 40%.

In some embodiments, the at least one of the AuPt particles has a size that is anywhere from 10 microns to 50 microns. Optionally, the at least one of the AuPt particles includes a plurality of pores, and the at least one of the pores has a pore size that is anywhere from 5 nanometers to 5 microns. Optionally, the at least one of the AuPt particles has a porosity that is anywhere from 5% to 30%.

In some embodiments, the at least one of the AuPt particles has a size that is anywhere from 20 microns to 40 microns. Optionally, the at least one of the AuPt particles comprises a plurality of pores, and wherein at least one of the pores has a pore size that is anywhere from 10 nanometers to 1 micron. Optionally, the at least one of the AuPt particles has a porosity that is anywhere from 10% to 20%.

In some embodiments, the platinum in the AuPt particles is in a range of 10-50% by weight. Optionally, the platinum in the AuPt particles is in a range of 20-40% by weight. Optionally, the platinum in the AuPt particles is in a range of 25%-35% by weight. Optionally, the platinum in the AuPt particles is 29% by weight.

Optionally, the AuPt particles comprise a volume magnetic susceptibility from −40×10⁻⁶ to 40×10⁻⁶.

Optionally, the AuPt particles are configured to provide radiopacity for the embolic fluid.

Optionally, wherein the AuPt particles are configured to allow use of magnetic resonance imaging (MRI) without magnetic resonance artifact.

A method of occluding an aneurysm includes: delivering an embolic agent comprising gold-platinum alloy (AuPt) particles to a target location inside a body of a patient, the target location comprising the aneurysm; and occluding the aneurysm by the embolic agent.

Optionally, the embolic agent is in a fluid form. Optionally, the embolic agent includes a gel, a paste, or a liquid solution. Optionally, the embolic agent includes a polymeric solution.

In some embodiments, the aneurysm comprises a neurovascular aneurysm. Optionally, the act of delivering the embolic agent includes delivering the embolic agent to a vasculature of a brain. Optionally, the act of delivering the embolic agent to the vasculature of the brain is performed by a delivery catheter.

Additionally, the method of occluding an aneurysm includes reducing blood flow by a balloon catheter, wherein the delivery catheter is configured to extend through the balloon catheter.

In some embodiments of the method of occluding an aneurysm, the platinum in the AuPt particles is in a range of 10-50% by weight. Optionally, the platinum in the AuPt particles is in a range of 20-40% by weight. Optionally, the platinum in the AuPt particles comprise 29% by weight. Optionally, the AuPt particles comprise a volume magnetic susceptibility from −40×10⁻⁶ to 40×10⁻⁶.

In some embodiments of the method of occluding an aneurysm, the embolic agent delivered to the target location, at least one of the AuPt particles has a size that is anywhere from 0.1 micron to 100 microns. Optionally, the embolic agent delivered to the target location, the at least one of the AuPt particles includes a plurality of pores, and the at least one of the pores has a pore size that is anywhere from 1 nanometer to 10 microns. Optionally, the embolic agent delivered to the target location, the at least one of the AuPt particles has a porosity that is anywhere from 1% to 40%.

In some embodiments of the method of occluding an aneurysm, the embolic agent delivered to the target location, at least one of the AuPt particles has a size that is anywhere from 10 microns to 50 microns. Optionally, the embolic agent delivered to the target location, the at least one of the AuPt particles includes a plurality of pores, and the at least one of the pores has a pore size that is anywhere from 5 nanometers to 5 microns. Optionally, the embolic agent delivered to the target location, at least one of the AuPt particles has a porosity that is anywhere from 5% to 30%.

In some embodiments of the method of occluding an aneurysm, the embolic agent delivered to the target location, at least one of the AuPt particles has a size that is anywhere from 20 microns to 40 microns. Optionally, the embolic agent delivered to the target location, the at least one of the AuPt particles includes a plurality of pores, and the at least one of the pores has a pore size that is anywhere from 10 nanometers to 1 micron. Optionally, the embolic agent delivered to the target location, the at least one of the AuPt particles has a porosity that is anywhere from 10% to 20%.

A vaso-occlusive agent for occluding an aneurysm including: an embolic fluid configured to occlude the aneurysm, and a plurality of radiopaque particles suspended within the embolic fluid, wherein at least one of the plurality of radiopaque particles has a volume magnetic susceptibility from −40×10⁻⁶ to 40×10⁻⁶.

Other and further aspects and features of embodiments will become apparent from the ensuing detailed description in view of the accompanying figures.

### BRIEF DESCRIPTION OF THE DRAWINGS

The drawings illustrate the design and utility of preferred embodiments of the disclosed inventions, in which similar elements are referred to by common reference numerals. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention, which is defined only by the appended claims and their equivalents. In addition, an illustrated embodiment of the disclosed inventions needs not have all the aspects or advantages shown. Further, an aspect or an advantage described in conjunction with a particular embodiment of the disclosed inventions is not necessarily limited to that embodiment and can be practiced in any other embodiments even if not so illustrated.

In order to better appreciate how the above-recited and other advantages and objects of the disclosed inventions are obtained, a more particular description of the disclosed inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is perspective view of a vial containing a vaso-occlusive agent in accordance with embodiments of the disclosed inventions;

FIGS. 2A-2B are close-up and cross-sectional views of radiopaque particles of the vaso-occlusive agent of FIG. 1;

FIG. 3 is an image of a post-procedure MRI of an aneurysm occluded with a prior art embolic agent; and

FIG. 4 is an image of a post-procedure MRI of an aneurysm occluded with a vaso-occlusive agent in accordance with embodiments of the disclosed inventions.

### DETAILED DESCRIPTION

The present disclosure is directed to vaso-occlusive treatment using liquid embolic solutions within the vasculature of a patient (e.g., within an aneurysmal sac). The liquid embolic solutions described herein have a low magnetic susceptibility to minimize visualization obscuring artifacts created during the post-procedure MRI (i.e., MRI compatible).

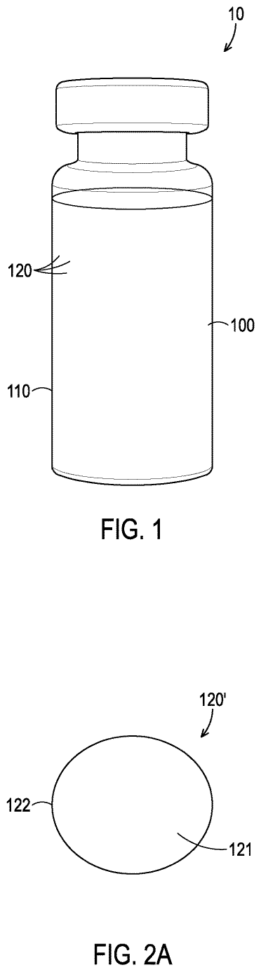

FIG. 1 illustrate a vial 10 containing a vaso-occlusive agent 100 in accordance with one embodiment of the disclosed inventions. The vaso-occlusive agent 100 is configured to occlude aneurysms in a target site or body tissue of a patient. In some embodiments, the target body tissue is a neurovascular aneurysm. Delivering the vaso-occlusive agent 100 to the target body tissue includes introducing the agent 100 into the vasculature of the brain with commonly used delivery catheter systems.

The vaso-occlusive agent 100 includes a mixture of polymers, adhesives, and radiopaque particles composed of metals and/or alloys, among other suitable biocompatible components. In one embodiment, the vaso-occlusive agent 100 includes an embolic agent 110 (e.g., embolic fluid 110) and a plurality of radiopaque particles 120 dispersed, suspended, diffused, distributed or added into the embolic fluid 110. The embolic fluid 110 may be a gel, paste, or liquid solution, where the embolic fluid 110 is composed of biocompatible materials. The biocompatible materials that made the embolic fluid 110 include polymers and/or adhesives. In some embodiments, some or substantially all of the biocompatible materials may have fast polymerization properties. For example, the embolic fluid 110 may include n-butyl cyanoacrylate (n-BCA), ethylene vinyl-alcohol copolymer (EVOH) and/or dimethyl-sulfoxide (DMSO), or any other suitable polymeric and/or adhesive materials.

Further, FIG. 1 shows the plurality of radiopaque particles 120 dispersed within the embolic fluid 110 for illustration purposes. However, it should be appreciated that the plurality of radiopaque particles 120 suspended within the embolic fluid 110 may not be visible to the naked eye, yet still present in the vaso-occlusive agent 100. The radiopaque particles 120 of the vaso-occlusive agent 100 are composed of metals, alloys and/or combinations of metallic materials, where the materials have radio-opaque properties for visualization (e.g., post-procedure visualization). The plurality of radiopaque particles 120 may be composed of a suitable biocompatible and radio-opaque material, such as tantalum, tantalum oxide, tungsten, palladium, barium sulfate, platinum, gold, iridium, alloys or other metals. In one embodiment, the plurality of radiopaque particles 120 is composed of a gold-platinum (AuPt) alloy (e.g., AuPt29), which has been demonstrated to have suitable radiopacity and MRI compatibility, as disclosed in U.S. Patent Publication No. 2020/0170647, entitled “Vaso-Occlusive Device”, which is fully incorporated herein by reference in its entirety.

In one embodiment, the plurality of radiopaque particles 120 are composed of gold-platinum (AuPt) alloy, where platinum in the AuPt alloy is within a range 10% to 50% by weight. For example, the platinum in the AuPt alloy may be in a range 20% to 40% by weight or in a range of 25% to 35% by weight. In one advantageous embodiment, platinum in the AuPt alloy is 29% by weight (i.e., AuPt 29) in the plurality of radiopaque particles 120. The volume magnetic susceptibility can be from −40×10⁻⁶ to 40×10⁻⁶, preferably from −20×10⁻⁶ to 20×10⁻⁶, more preferably−20×10⁻⁶ to 10×10⁻⁶. In the embodiments where the plurality of radiopaque particles 120 are composed of AuPt29, the AuPt29 alloy includes a volume magnetic susceptibility of about −9×10⁻⁶, which is similar to the magnetic susceptibility of human tissue. Therefore, the AuPt29 alloy particles 120 are well suited for MRI compatibility (e.g., avoiding and/or substantially minimizing MRI artifact), which will be described in further detail below.

In one embodiment, the percentage of the plurality of radiopaque particles 120 suspended in the embolic fluid 110 is between 10% to 50% by weight. In an advantageous embodiment, the percentage of the plurality of radiopaque particles 120 suspended in the embolic fluid 110 is between 20% to 30% by weight.

The plurality of radiopaque particles 120 can be manufactured by atomization process, where the raw materials (i.e., gold and platinum) constituting the desirable AuPt alloy composition are weighed and mixed. After degassing the molten metal, the pre-mixed raw materials are melted to form a liquid or molten metal that is poured into a nozzle. The molten metal coming from the nozzle is disintegrated (i.e., atomize) into significantly small droplets by a high-pressure gas or water stream. The droplets are cool down rapidly forming the plurality of radiopaque particles 120. The particles size can be controlled by the size of the nozzles used and pressure of the gas or water passing through the nozzle. Alternatively, the plurality of radiopaque particles 120 can be manufactured as porous AuPt particles by changing the mixing ratio between the molten metal and the gas, therefore, trapping gas bubbles which become pores after the molten metal solidifies. Additionally, the AuPt particles with nano/submicron pores may be manufactured by the above-described atomization process to produce ternary AuPtAl particles of desirable composition. Then, leach out the Al from AuPtAl particles by placing the AuPtAl particles into an alkaline solution (e.g., NaOH), producing the nano/submicron pores on the AuPt particles. Further, the porous AuPt particles may be filtered out from the solution, rinsed, and dried to be combined and suspended in the embolic fluid 110 of the vaso-occlusive agent 100.

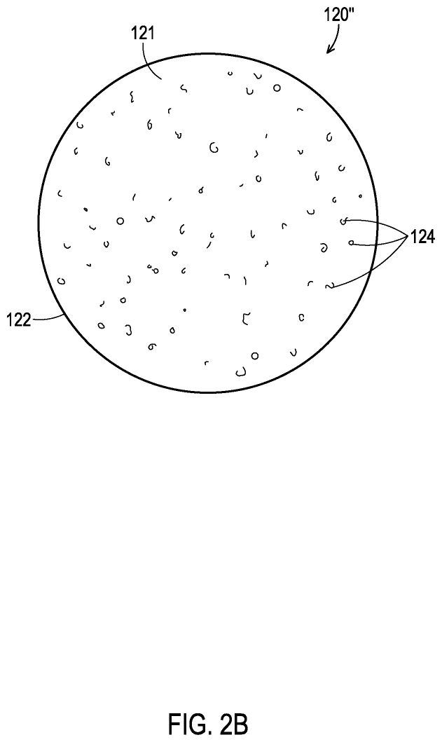

FIGS. 2A-2B illustrate close-up and cross-sectional views of AuPt particles 120′ and 120″ the plurality of radiopaque particles contained in the vaso-occlusive agent 100 of FIG. 1. It should be appreciated that the features of the AuPt particle 120′ and/or 120″ described in FIGS. 2A-2B are the same features of substantially all, some or at least one of the plurality of radiopaque particles 120. The AuPt particles 120′ and 120″ may have a round, spherical, elliptical, ovoid or any other suitable configuration. The AuPt particles 120′ and 120″ have a size that is anywhere from 0.1 micron to 100 microns. In one embodiment, each of the AuPt particles 120′ and 120″ have a size in a range between 10 to 50 microns. In another embodiment, each of the AuPt particles 120′ and 120″ have a size in a range between 20 to 40 microns.

The AuPt particle 120′ includes an inner portion 121, where the inner portion is substantially solid (i.e., void of pores) and an outer surface 122 of the particle 120', as shown in FIG. 2A. While the AuPt particle 120″ includes a plurality of pores 124 within the inner portion 121 and/or in the outer surface 122 of the particle 120″, as shown in FIG. 2B. The pores 124 may have a pore size that is anywhere from 1 nanometer to 10 microns, where substantially all, some or at least one of the pores 124 have a pore size in a range between 1 nanometer to 10 microns. In one exemplary embodiment, when the AuPt particle 120″ has a size from 1 to 100 microns, at least one of the pores 124 have a pore size of 1 nanometer to 10 microns, and the percentage of the plurality of radiopaque particles 120 suspended in the embolic fluid 110 is between 1% to 40% by weight. In another embodiment, when the AuPt particle 120″ has a size from 10 to 50 microns, at least one of the pores 124 have a pore size of 5 nanometers to 5 microns, and the percentage of the plurality of radiopaque particles 120 suspended in the embolic fluid 110 is between 5% to 30% by weight. In an advantageous embodiment, when the AuPt particle 120″ has a size from 20 to 40 microns, at least one of the pores 124 have a pore size of 10 nanometers to 1 micron, and the percentage of the plurality of radiopaque particles 120 suspended in the embolic fluid 110 is between 10% to 20% by weight. The advantageous embodiment includes a combination of low sedimentation and radiopacity for the vaso-occlusive agent 100.

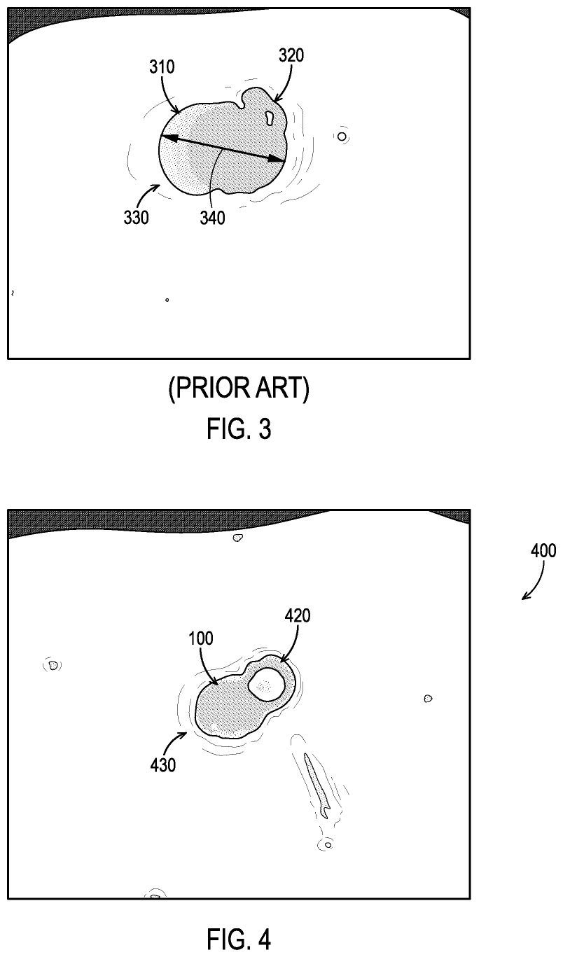

FIG. 3 is an image of a post-procedure MRI 300 of an aneurysm 330 occluded with a prior art embolic agent 310. The embolic agent 310 is composed of commercially available Pt/8W, which may include embolic coils (not shown). The volume magnetic susceptibility for Pt/8W is between 100×10⁻⁶ to 120×10⁻⁶, which is significantly higher than human tissue and therefore produces obscuring artifact 340. The obscuring artifact 340 is substantially large (e.g., increase of 80% to 100% in size). In some cases, the obscuring artifact 340 doubles the image size of the aneurysm 330. For example, the aneurysm 330 actual size is about 8 millimeters, while the image of the aneurysm 330 as shown in FIG. 3 is about 16 millimeters (see diameter line of 340). The obscuring artifact 340 produces undesirable results, such as, distorted and disproportionated large images of the aneurysm 330 and further obscures the view of elements disposed near or adjacent to the aneurysm 330 (e.g., silicon tube 320). The undesirable obscuring artifact of the emboli agent 310 is even worse when the embolic agent contains tantalum. The volume magnetic susceptibility for Ta is 178×10⁻⁶, which produces adverse and significantly larger obscuring artifact.

FIG. 4 is an image of a post-procedure MRI 400 of an aneurysm 430 occluded with the vaso-occlusive agent 100 of FIGS. 1-2B, in accordance with embodiments of the disclosed inventions. The embolic agent 100 is composed of AuPt29, which may include embolic coils (not shown). MRI is typically used to visualize the treatment site post-procedure (e.g., a few weeks after initial treatment of the aneurysm) to ensure that the aneurysm is properly occluded. The vaso-occlusive agent 100 is composed in a manner that enables radiopacity during treatment of the aneurysm, while avoiding or minimizing visualization obscuring artifacts created during the post-procedure MRI. For example, the vaso-occlusive agent 100 of FIG. 4 includes the plurality of radiopaque particles 120 composed of AuPt29. The AuPt29 alloy has a volume magnetic susceptibility of about −9×10⁻⁶, which is similar to the magnetic susceptibility of human tissue. Therefore, the vaso-occlusive agent 100 containing the AuPt29 alloy particles 120 suspended in the embolic fluid 110 (FIGS. 1-2B) is well suited for MRI compatibility, avoiding and/or minimizing visualization obscuring artifacts (e.g., minimal to negligent MR artifact). As shown in FIG. 4, the image size of the aneurysm 430 is the same or substantially similar to the actual size of the aneurysm (e.g., 8 millimeters) and the image is not distorted. Additionally, the elements disposed near or adjacent to the aneurysm 430 (e.g., silicon tube 420) are visible (e.g., not obscured).

Although particular embodiments have been shown and described herein, it will be understood by those skilled in the art that they are not intended to limit the disclosed inventions, and it will be obvious to those skilled in the art that various changes, permutations, and modifications may be made (e.g., the dimensions of various parts, combinations of parts) without departing from the scope of the disclosed inventions, which is to be defined only by the following claims and their equivalents. The specification and drawings are, accordingly, to be regarded in an illustrative rather than restrictive sense. The various embodiments shown and described herein are intended to cover alternatives, modifications, and equivalents of the disclosed inventions, which may be included within the scope of the appended claims.

What is claimed is:

1. A vaso-occlusive agent for treatment of a vessel defect, the agent comprising:
an embolic fluid configured to treat the vessel defect;
a plurality of gold-platinum alloy (AuPt) particles suspended within the embolic fluid;
wherein each of the AuPt particles are formed of an AuPt alloy comprising platinum in a range of 25%-35% by weight; and
wherein each of the AuPt particles has a plurality of pores such that each of the AuPt particles has a porosity that is anywhere from 5% to 30%.

2. The vaso-occlusive agent of claim 1, wherein the AuPt particles are formed of an AuPt alloy comprising approximately 29% platinum by weight.

3. The vaso-occlusive agent of claim 1, wherein each of the AuPt particles has a size that is anywhere from 0.1 micron to 100 microns.

4. The vaso-occlusive agent of claim 3, wherein the pores have a pore size that is anywhere from 1 nanometer to 10 microns.

5. The vaso-occlusive agent of claim 1, wherein the embolic fluid comprises a gel, a paste, or a liquid solution.

6. The vaso-occlusive agent of claim 5, wherein the embolic fluid comprises a biocompatible material comprising ethylene vinyl-alcohol copolymer and dimethyl-sulfoxide.

7. The vaso-occlusive agent of claim 1, wherein the vessel defect is an aneurysm.

8. The vaso-occlusive agent of claim 1, wherein the AuPt particles have a volume magnetic susceptibility in a range from −20×10⁻⁶ to 10×10⁻⁶.

9. The vaso-occlusive agent of claim 1, wherein the AuPt particles are configured to provide radiopacity for the embolic fluid.

10. The vaso-occlusive agent of claim 1, wherein the AuPt particles are configured to allow use of magnetic resonance imaging (MRI) without magnetic resonance artifact.

11. A method of treating a vessel defect, comprising:
delivering an embolic agent to a target location inside a body of a patient, the target location comprising the vessel defect, the embolic agent comprising:
an embolic fluid configured to treat the vessel defect;
a plurality of gold-platinum alloy (AuPt) particles suspended within the embolic fluid;
wherein each of the AuPt particles are formed of an AuPt alloy having platinum in a range of 25%-35% by weight; and
wherein each of the AuPt particles has a plurality of pores such that each of the AuPt particles has a porosity that is anywhere from 5% to 30%; and
treating the vessel defect by the embolic agent.

12. The method of claim 11, wherein the AuPt particles are formed of an AuPt alloy comprising approximately 29% platinum by weight.

13. The method of claim 11, wherein each of the AuPt particles has a size that is anywhere from 0.1 micron to 100 microns.

14. The method of claim 13, wherein the pores have a pore size that is anywhere from 1 nanometer to 10 microns.

15. The method of claim 11, wherein the embolic agent comprises a gel, a paste, or a liquid solution.

16. The method of claim 15, wherein the embolic fluid comprises a biocompatible material comprising ethylene vinyl-alcohol copolymer and dimethyl-sulfoxide.

17. The method of claim 1, wherein the vessel defect is an aneurysm and treating the vessel defect comprises occluding the aneurysm.

18. The method of claim 11, wherein the AuPt particles have a volume magnetic susceptibility in a range from −20×10⁻⁶ to 10×10⁻⁶.

19. The method of claim 11, wherein the AuPt particles are configured to provide radiopacity for the embolic fluid.

20. The method of claim 11, wherein the AuPt particles are configured to allow use of magnetic resonance imaging (MRI) without magnetic resonance artifact.
